(19) United States
(12) Patent Application Publication
(10) Pub. No.: US 2021/0059101 A1
(43) Pub. Date: Mar. 4, 2021

(54) A Steam Disinfection Inline System and Apparatus Agricultural Particulate Products

(71) Applicant: The State of Israel, Ministry of Agriculture & Rural Development Agricultural Research Organization, Rishon Lazion (IL)

(72) Inventors: Rafi REGEV, Rishon Le Tsiyon (IL); Ze'ev SCHMILOVITCH, Yehud (IL); Ahron HOFFMAN, Giv'atayim (IL); Dani ESHEL, Pardes Hanna-Karkur (IL); Dekel MEIR, Midreshet Ben Gurion (IL); Elad RIMON, Tel Aviv (IL)

(21) Appl. No.: 16/963,601

(22) PCT Filed: Jan. 21, 2019
(86) PCT No.: PCT/IB2019/050483

§ 371 Date: Jul. 21, 2020

Related U.S. Application Data
(60) Provisional application No. 62/619,823, filed on Jan. 21, 2018.

(51) Int. Cl. A01C 1/08; A61L 2/00; A61L 2/26; A61L 2/24

(52) U.S. Cl. CPC A01C 1/08; A61L 2/0023; A61L 2/26; A61L 2202/14; A61L 2202/122; A61L 2202/15; A61L 2/24

(57) ABSTRACT

The present invention provides systems and methods for treating agricultural particulates, such as seeds, beans, granular and powder agricultural products, and the like, with a treatment agent, such as steam, as the agricultural products move forward along a pathway created by an auger, in a chamber, enclosed by an aligned celling and floor, and oppositely disposed lateral walls.

### CROSS-REFERENCES TO RELATED APPLICATIONS

This application is related to and claims priority from commonly owned U.S. Provisional Patent Application Ser. No. 62/619,823, entitled: A Steam Disinfection Inline System and Apparatus for Agricultural and Other Products in Their Granular or Powdery Form, filed on Jan. 21, 2018, the disclosure of which is incorporated by reference in its entirety herein.

### TECHNICAL FIELD

The present invention relates to the disinfection of agricultural and other products in their particulate, granular or powdery form.

### BACKGROUND OF THE INVENTION

There is an increasing public concern over the level of pesticide residues in agricultural products. Many key fungicides which have heretofore been used have resulted in the development of pathogen resistance to such fungicides. As a result, the use of pesticides and fungicides for post-harvest control of diseases in agricultural products has been substantially restricted and considerable research and development has been conducted for using other post-harvest treatments of agricultural products to extend their storage life.

Heat treatment for disinfection leaves no pesticide residues and is considered as both environmental friendly and human friendly. However, when heat is applied to the complete outer surface of the agricultural products sufficient for post-harvest disease control, there is a danger that hot spots may be produced on the outer surface of the agricultural products, damaging them, or making them particularly vulnerable to necrotrophic pathogens.

### SUMMARY OF THE INVENTION

The present invention provides systems and methods for treating agricultural particulates, such as seeds, beans, granular and powder agricultural products, and the like, with a treatment agent, such as steam, as the agricultural products move forward along a pathway created by an auger, in a chamber, enclosed by an aligned celling and floor, and oppositely disposed lateral walls. The auger rotates at speeds sufficient to cause the agricultural particulates to leave the auger surface, such that substantially all of the exteriors of the agricultural particulates are exposed to the steam. The exposure times to the steam, created by the intervals and lengths in which steam enters the chamber through which the auger extends, coupled with rotational speed of the auger, and the temperature of the steam, results in the elimination (destruction) and/or neutralization of the pathogens on the agricultural particulates, while the agricultural particulates remain undamaged and active.

Embodiments of the invention are directed to a disinfection method comprising: placing a plurality of particulates along a rotational pathway for causing movement of the particulates to expose the exterior surface of said particulates to treatment substances; providing a treatment chamber along the rotational pathway; and, treating the particulates with a treatment substance while the particulates are passing through said treatment chamber.

Optionally, the method is such that the treatment substance is steam.

Optionally, the method is such that the treatment substance is a treatment agent.

Optionally, the method is such that the rotational pathway is created by a rotating auger.

Optionally, the method is such that the particulates include seeds.

Optionally, the method is such that the particulates are in a powdery form.

Optionally, the method is such that the size of the particulates ranges approximately between 25 μm and 20 mm.

Optionally, the method is such that the exposure period of the particulates to the treatment substance ranges approximately between 0-20 seconds.

Optionally, the method additionally comprises introducing the steam into the treatment chamber through at least one nozzle in the treatment chamber.

Optionally, the method is such that the steam is introduced through said at least one nozzle at a temperature of approximately 120° 440° C.

Optionally, the method is such that the steam causes the particulates surface temperature to reach a temperature of approximately 70° C.

Embodiments of the invention are directed to a system for treating agricultural particulates. The system comprises: a chamber configured for receiving a treatment agent in the interior of the chamber; an auger extending at least partially through the chamber, the auger configured for maintaining a forward moving path for the agricultural particulates, and the auger is rotatable at speeds for causing the particulates to move off of the surface of the auger such that substantially all of exterior of the particulates is exposed to the treatment agent.

Optionally, the system additionally comprises a steam source, for providing steam as the treatment agent to the interior of the chamber.

Optionally, the system additionally comprises a computerized controller configured for controlling the stream source, such that steam is provided to the chamber at predetermined intervals.

Optionally, the system is such that the computerized controller is configured for controlling the stream source, such that steam is provided to the chamber at the predetermined intervals for predetermined lengths of time.

Optionally, the system is such that the chamber includes an opening for receiving the agricultural particulates at a proximal end of the chamber, and an opening through which the agricultural particulates are discharged from the chamber at a distal end.

Optionally, the system is such that the forward moving path is from the opening at the proximal end of the chamber to the opening at the distal end of the chamber.

Optionally, the system is such that the auger is driven by a motor.

Optionally, the system is such that the motor is controlled by the computerized controller.

Optionally, the system is such that the chamber is formed of a floor aligned with a ceiling and laterally disposed walls between the floor and the ceiling, the floor and laterally disposed walls in proximity to the edges of the auger for maintaining the forward moving path for the agricultural particulates.

Optionally, the system is such that the opening at the proximal end of the chamber extends through the ceiling, and the opening at the distal end of the chamber extends through the floor.

Optionally, the system is such that the opening at the distal end of the chamber is in communication with a draining conveyer.

Unless otherwise defined herein, all technical and/or scientific terms used herein have the same meaning as commonly understood by one of ordinary skill in the art to which the invention pertains. Although methods and materials similar or equivalent to those described herein may be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the present invention are herein described, by way of example only, with reference to the accompanying drawings. With specific reference to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Attention is now directed to the drawings, where like reference numerals or characters indicate corresponding or like components.

In the drawings:

FIG. 1A is a side view of the system according to an embodiment of the present invention; and,

FIG. 1B is a top view of the system according to an embodiment of the present invention.

### DESCRIPTION OF THE PREFERRED EMBODIMENTS

The present invention is not limited in its application to the details of construction and the arrangement of the components set forth in the following description. The invention is capable of other embodiments, or of being practiced or carried out in various ways. Also, it is to be understood that the phraseology and terminology employed herein is for the purpose of description and should not be regarded as limiting.

The present invention provides a system for disinfecting agricultural products, as particulates, such as seeds, beans, granular and powder agricultural products, and the like, from potential pathogens using short and efficient exposure to steam.

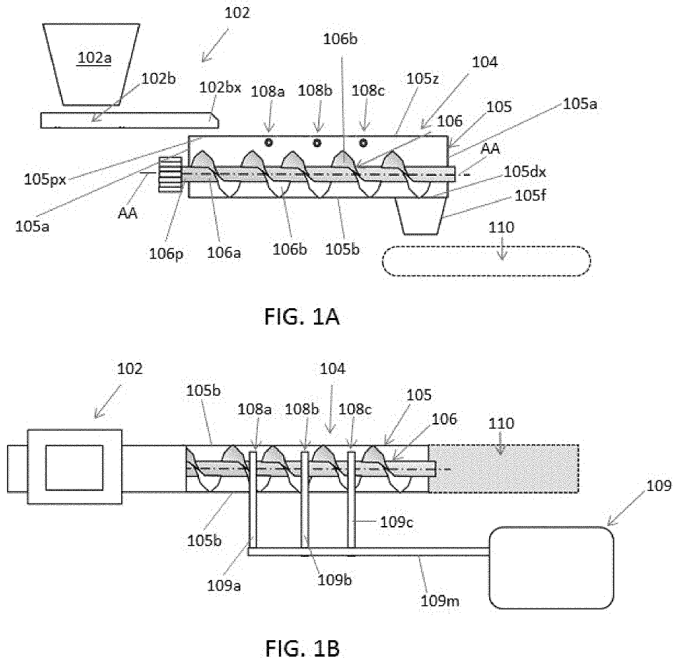

FIGS. 1A and 1B are respective side and top views of the system 100 for treating agricultural products, including agricultural particulates or particulates, such as seeds, beans, granular and powder agricultural products, and the like, hereinafter and collectively known as “agricultural particulates” or “particulates”, these terms used interchangeably herein. The system 100 includes a feeder 102, a treatment unit 104 and a draining conveyer 110 in alignment with each other, to form a pathway for the particulates, from a raw product to a treated product.

The feeder 102 includes a hopper 102a, e.g., a vibrating hopper, which is aligned over a conveyer 102b, e.g., a belt conveyer, onto which the particulates fall. The end 102bx is positioned over a proximal opening 105px of a chamber 105 or enclosure of the treatment unit 104.

The treatment unit 104 is such that the chamber 105 encloses an auger 106. The auger 106 is formed of an axial portion 106a (extending along an axis AA), and a helical protrusion 106b, extending along the length of the axial portion 106a. A proximal portion of the auger 106p extends out of the chamber 105, so as to be driven by a motor (not shown) via gears or belts. The motor may be activated manually or controlled by a computerized controller (not shown), over wired and/or wireless networks, or combinations thereof. The helical protrusion 106b extends from the axial portion 106a (this extension defining a diameter for the helical protrusion 106b) to proximate to the lateral 105a and lower walls (floor) 105b of the chamber 105, and coupled with the rotation of the auger 106, forms a forward path for the particulates, from the proximal opening 105px to the distal opening 105dx of the chamber 105. This configuration prevents particulates from moving backward (toward the proximal opening 105px), in the direction opposite the forward direction (toward the distal opening 105dx). The chamber 105 includes a distal opening 105dx in the floor 105b of the chamber 105, this opening 105dx aligning with a funnel member 105f, through which the particulates are deposited onto the draining conveyer 110.

The chamber 105 includes one or more nozzles 108a-108c, for example, in the chamber upper walls 105z, through which steam enters the chamber 105, from a steam unit or source 109, via pipes 109a-109c (from a main pipe 109m). The steam unit or source 109 may be controlled by a computer, computerized controller or the like, as well as manually.

The computer or manual control is such that steam, which is for example, water based, is released from the steam unit 109 at various times, including time intervals, for various timed lengths. The steam is at a temperature sufficient, such that coupled with the auger 106 rotation speed, which causes the particulates to move off of the surface of the auger 106 by rotating and/or turning, such that all or substantially all, of the exterior (or surface) of the particulates is exposed to the steam in the chamber 105. The exposure to the steam eliminates and/or neutralizes pathogens for each of the particulates, for example, disinfecting each of the particulates without destroying or deactivating the particulates themselves.

In addition to steam, other treatment substances or agents, either alone or in combination with steam, may also be transmitted into the chamber 105 for treatment of the particulates. The treatment agents, are typically gasses or liquids, but may also be solids, such as powders, for example, which are food safe, or UV light.

The draining conveyer 110 is, for example, a screening conveyer, which receives the particulates from the treatment unit 104 and is used for draining and collecting the treated particulates.

For example, the system 100 is designed to accommodate particulates (and other materials) of between approximately 25 μm and 20 mm. The system 100 is also suited for larger agricultural products, such as, sunflower seeds which are greater than 20 mm in length and/or diameter.

In operation, for example, the steam enters the chamber 105 through the steam nozzles 108, at temperatures of approximately 120° C.-140° C. This entry temperature is such that the steam contacts the particulates surfaces at temperatures sufficient to treat the particulates from pathogens and other harmful agents, but not cause heat damage to the particulates. The steam causes the surface temperature of the particulates to reach a temperature of approximately 70° C., as this temperature allows disinfecting of the particulates from pathogens and other harmful agents without causing heat damage to the particulates (e.g., such that seeds remain active). The temperature of the steam declines due to the distance between the steam nozzles 108a-108c and the particulates on the auger 106. The exposure time of the particulates to the steam is, for example, restricted to a predetermined period of time, approximately 0-20 seconds, as provided by controlling the auger 106 rotational speed, manually or by a computer controller, and/or coupled with the exposure time to the steam, in order to prevent heat damage to the particulates.

### EXAMPLES

The following examples are not meant to limit the scope of the claims in any way. The following examples are put forth so as to provide those of ordinary skill in the art with a complete disclosure and description of how to make and use the described invention, and are not intended to limit the scope of the invention, nor are they intended to represent that the experiments below are all or the only experiments performed. Unless indicated otherwise, parts are parts by weight, molecular weight is weight average molecular weight, temperature is in degrees Centigrade, and pressure is at or near atmospheric.

### Example 1—Disinfecting Sesame Seeds

Two types of sesame seeds, raw and roasted, were tested for the total number of pathogens. The seeds were exposed to steam for various exposure times. The exposure times were: control—0 seconds, treatment periods—10, 15 and 20 seconds.

| | Exposure time | Total count | Total count |
|---|---|---|---|
| | (sec) | Repetition 1 | Repetition 2 |
| Roasted seeds | 0 | 7000 | 10250 |
| | 10 | 4500 | 650 |
| | 15 | 450 | 710 |
| | 20 | 530 | 420 |
| Raw seeds | 0 | 10000 | 12000 |
| | 10 | 780 | 810 |
| | 15 | 810 | 350 |
| | 20 | 900 | 680 |

The exposure to steam of both raw and roasted sesame seeds has provided a highly disinfected seeds with much lesser number of pathogens.

### Example 2—Disinfecting Seeds of Raw Materials Used for Salad Spreads

Three types of seeds of raw materials (eggplant, pepper, red cabbage) used for salad spreads were tested for the total number of pathogens, as well as, for the specific number of three specific pathogens (E. coli, yeasts, and coliforms). The raw materials were exposed to steam for various exposure times. The exposure times were: control—0 seconds, treatment periods—10, 15 and 20 seconds.

| | Exposure time | | | | |
|---|---|---|---|---|---|
| Product | (Sec) | Total count | E. Coli | Yeats | Coliforms |
| Eggplant | 0 | 650 | <10 | <10 | 140 |
| | 10 | <10 | <10 | <10 | <10 |
| | 20 | <10 | <10 | <10 | <10 |
| Pepper | 0 | 3450 | <10 | <10 | 115 |
| | 10 | <10 | <10 | <10 | <10 |
| | 20 | <10 | <10 | <10 | <10 |
| Red Cabbage | 0 | 10000 | 150 | 110 | 8500 |
| | 15 | <10 | <10 | 50 | <10 |
| | 20 | <10 | <10 | <10 | <10 |

The exposure to steam of the seeds of the raw materials (eggplant, pepper, red cabbage) has provided highly disinfected raw materials' seeds with a significantly lesser number of pathogen.

While the invention has been described with respect to a limited number of embodiments, it will be appreciated that many variations, modifications and other applications of the invention may be made. Therefore, the claimed invention as recited in the claims that follow is not limited to the embodiments described herein.

1. A disinfection method comprising:
placing a plurality of particulates along a rotational pathway for causing movement of said particulates to expose the exterior surface of said particulates to treatment substances;
providing a treatment chamber along said rotational pathway; and,
treating said particulates with a treatment substance while said particulates are passing through said treatment chamber.

2. The method of claim 1, wherein said treatment substance is steam.

3. The method of claim 1, wherein said treatment substance is a treatment agent.

4. The method of claim 1, wherein said rotational pathway is created by a rotating auger.

5. The method of claim 1, wherein said particulates include seeds.

6. The method of claim 1, wherein said particulates are in a powdery form.

7. The method of claim 1, wherein the size of said particulates ranges approximately between 25 μm and 20 mm.

8. The method of claim 1, wherein the exposure period of said particulates to said treatment substance ranges approximately between 0-20 seconds.

9. The method of claim 2, additionally comprising introducing said steam into said treatment chamber through at least one nozzle in the treatment chamber.

10. The method of claim 8, wherein said steam is introduced through said at least one nozzle at a temperature of approximately 1200-1400 C.

11. The method of claim 8, wherein said steam causes the particulates surface temperature to reach a temperature of approximately 700 C.

12. A system for treating agricultural particulates comprising:
a chamber configured for receiving a treatment agent in the interior of the chamber;
an auger extending at least partially through the chamber, the auger configured for maintaining a forward moving path for the agricultural particulates, and the auger is rotatable at speeds for causing the particulates to move off of the surface of the auger such that substantially all of exterior of the particulates is exposed to the treatment agent.

13. The system of claim 12, additionally comprising a steam source for providing steam as the treatment agent to the interior of the chamber.

14. The system of claim 13, additionally comprising a computerized controller configured for controlling the stream source, such that steam is provided to the chamber at predetermined intervals.

15. The system of claim 14, wherein the computerized controller is configured for controlling the stream source, such that steam is provided to the chamber at the predetermined intervals for predetermined lengths of time.

16. The system of claim 12, wherein the chamber includes an opening for receiving the agricultural particulates at a proximal end of the chamber, and an opening through which the agricultural particulates are discharged from the chamber at a distal end.

17. The system of claim 16, wherein the forward moving path is from the opening at the proximal end of the chamber to the opening at the distal end of the chamber.

18. The system of claim 12, wherein the auger is driven by a motor.

19. The system of claim 18, wherein the motor is controlled by the computerized controller.

20. The system of claim 16, wherein the chamber is formed of a floor aligned with a ceiling and laterally disposed walls between the floor and the ceiling, the floor and laterally disposed walls in proximity to the edges of the auger for maintaining the forward moving path for the agricultural particulates.

21. The system of claim 20, wherein the opening at the proximal end of the chamber extends through the ceiling, and the opening at the distal end of the chamber extends through the floor.

22. The system of claim 21, wherein the opening at the distal end of the chamber is in communication with a draining conveyer.

23. The system of claim 14, wherein the auger is driven by a motor.
